(19) United States
(12) Patent Application Publication
(10) Pub. No.: US 2012/0107451 A1
(43) Pub. Date: May 3, 2012

(54) METHOD FOR PRODUCING A FERMENTED MILK PRODUCT

(75) Inventors: Ditte Marie Folkenberg, Hillerod (DK); Lone Poulsen, Rodovre (DK)

(73) Assignee: CHR-HANSEN A/S

(21) Appl. No.: 13/381,215

(22) PCT Filed: Jun. 30, 2010
(86) PCT No.: PCT/EP2010/059303

§ 371 Date: Dec. 28, 2011

(30) Foreign Application Priority Data
Jun. 30, 2009 (DK) PA 2009 00815

(51) Int. Cl. A23C 9/13; A23C 9/12; A23K 1/00; A23C 9/133; A23C 9/137; C12N 1/20; A23C 9/127

(52) U.S. Cl. 426/43; 435/252.9; 435/253.4; 426/580; 426/61; 426/71

(57) ABSTRACT

The present invention relates to a method for producing a fermented milk product with eithanced gel stiffness, wherein a polysaccharide producing Lactobacillus, in particular L. johnsonii, is used as starter.

### FIELD OF INVENTION

The present invention relates to a method for producing a fermented milk product with enhanced gel stiffness.

### BACKGROUND OF INVENTION

Lactic acid bacteria are extensively used for production of fermented foods, and they greatly contribute to flavor, texture and overall characteristics of these products. An old and well known example is yoghurt which probably originated from the Middle East and which still makes up more than half of the fermented milk production—or approximately 19 million tons in 2008 (source: Euromonitor). Fermented milks as e.g. yoghurts are popular due to the healthy image and pleasant sensory properties.

In many parts of the world an increasing interest in low fat fermented milk products is seen. This poses significant challenges for lactic acid bacteria culture as well as for the production process because it is difficult to produce low fat fermented milk products without reduction of sensory quality.

Yoghurt is produced from milk that has been standardized with respect to fat and protein content, homogenized and heat treated. Hereafter the milk is inoculated with a culture of Streptococcus thermophilus and Lactobacillus delbrueckii subsp. bulgaricus and subsequently fermented to a pH of around 4.5. In addition to the traditional yoghurt culture, a probiotic culture, as e.g. Bifidobacterium, can be applied to add extra heath benefits.

Texture is a very important quality parameter for fermented milks. A smooth consistency with high mouthfeel and mouth coating is required by the consumers. The trend is that increased mouthfeel (viscosity) and mouth coating is requested—even in low fat fermented milk products. A high viscosity can be obtained in fermented milk products by the use of exopolysaccharide-producing lactic acid bacteria cultures. At the same time, is it also requested that the products have a high level of gel stiffness. A high level of gel stiffness gives a thick appearance of the product and resistance to the spoon when stirring prior to eating, which is well liked by many consumers. The gel stiffness in a fermented milk product is mainly governed by the strength/density of the protein network formed during acidification of the milk. Both exopolysaccharides and protein network is known to ensure protection again the common defect syneresis (whey separation on top of the product) during storage. The combination of high viscosity (exopolysaccharides) and high gel firmness can, however, be difficult to obtain in (additive free) yoghurts, as the presence of exopolysaccharides seems to physically inhibit the formation of a tight protein network.

The trend in many regions is that a mild flavor (low post acidification) with aromatic notes is the preferred flavor profile. A large part of the world's yoghurt production is, however, added flavors and/or fruit preparations.

New culture compounding techniques, such as use of species which are not traditionally applied for yoghurt production and/or interactions between bacteria species, are interesting in order to obtain these targets.

Thus, there is a need for improved fermented milk products and lactic acid bacteria cultures for production of these products.

### SUMMARY OF INVENTION

The present inventors have surprisingly found that a certain group of lactic acid bacteria has the ability to ferment milk, resulting in a fermented milk product with high viscosity, high gel stiffness, high mouth coating, pleasant flavor, and low post acidification, also when compared to traditional yoghurt.

Thus, in an important aspect, the present invention relates to the use of polysaccharide producing strains of the species Lactobacillus fermentum to replace (fully or partly) Lactobacillus delbrueckii subsp. bulgaricus strains in ‘yoghurt’ cultures to enhance gel stiffness and mouth coating in a fermented milk product while maintaining or enhancing high viscosity.

In further aspects, the present invention relates to starter cultures comprising the lactic acid bacteria, and to fermented milk products made by fermentation of milk with a starter culture of the invention.

### DETAILED DISCLOSURE

In a first aspect, the present invention relates to a method for producing a fermented milk product, comprising fermenting a milk substrate with a strain belonging to a Lactobacillus species, which is able to produce a polysaccharide and/or a glycosyltransferase enzyme; and/or with a strain belonging to the species Lactobacillus fermentum. Preferred glycosyltransferases in context of the present invention are fructosyl transferase and glucosyl transferase. The transferases belongs to group EC 2.4 of the enzyme classification system. Preferred polysaccharides in context of the present invention are exopolysaccharide, homopolysaccharide and heteropolysaccharide.

The method of the invention may further comprise fermenting the milk substrate with a strain belonging to the species: Streptococcus thermophilus, such as a polysaccharide producing strain, and/or a strain selected from the group consisting of: DSM22592, DSM22585, DSM18111, DSM21408, DSM22587, DSM22884, CNCM I-3617 (WO2008/040734), DSM18344 (WO2007/144770), and CNCM I-2980 (US2006/0240539), and mutants and variants of any of these.

The milk substrate may be fermented with a strain belonging to the species Streptococcus thermophilus before, during, or after the fermentation with a strain belonging to a Lactobacillus species. It is presently preferred that the milk substrate is fermented with a strain belonging to the species Streptococcus thermophilus during the fermentation with a strain belonging to a polysaccharide producing Lactobacillus species.

In an interesting embodiment, the method of the invention comprises adding an enzyme to the milk substrate before, during and/or after the fermenting, such as an enzyme selected from the group consisting of: an enzyme able to crosslink proteins, transglutaminase, an aspartic protease, chymosin, and rennet.

It is presently preferred that the Lactobacillus species is Lactobacillus fermentum. Most preferred is the strain Lactobacillus fermentum DSM22584, and mutants and variants of this strain.

In a further aspect, the present invention relates to a strain belonging to a polysaccharide (e.g. homopolysaccharide or a heteropolysaccharide) producing Lactobacillus species, such as a strain which comprises a nucleotide sequence encoding a glycosyltransferase (e.g. fructosyl transferase or glucosyl transferase) enzyme, and/or a strain which produces a glycosyltransferase (e.g. fructosyl transferase or glucosyl transferase) enzyme, and to a strain belonging to a polysaccharide (e.g. homopolysaccharide or a heteropolysaccharide) producing Lactobacillus species, said strain comprises a nucleotide sequence encoding a glycosyltransferase (e.g. fructosyl transferase or glucosyl transferase) enzyme, and/or the strain produces a glycosyltransferase (e.g. fructosyl transferase or glucosyl transferase) enzyme.

In an important embodiment, the bacterial strain is Lactobacillus fermentum DSM22584, and mutants and variants of this strain.

In another aspect, the present invention relates to a bacterial strain belonging to the species Streptococcus thermophilus, selected from the group consisting of: DSM22592, DSM22585, DSM18111, and DSM21408, DSM22587, DSM22884, and mutants and variants of any of these strains.

In yet another aspect, the present invention relates to a composition comprising, either as a mixture or as a kit-of-parts,
a strain belonging to a polysaccharide (such as a homopolysaccharide or a heteropolysaccharide) and/or glycosyltransferase (e.g. fructosyl transferase or glucosyl transferase) enzyme producing Lactobacillus species; and
a strain belonging to the species Streptococcus thermophilus (such as a polysaccharide producing strain).

In an important embodiment, the composition of the invention comprises at least 10exp7 CFU (cell forming units) of a strain belonging to a polysaccharide and/or glycosyltransferase enzyme producing Lactobacillus species; and at least 10exp8CFU of a strain belonging to the species Streptococcus thermophilus.

The composition of the invention may be usable as a starter culture, and may be in frozen, freeze-dried or liquid form.

A presently preferred embodiment is a composition of the invention, wherein the strain belonging to the Lactobacillus species is selected from the group consisting of Lactobacillus fermentum DSM22584, and mutants or variants of this strain; and the strain belonging to the species Streptococcus thermophilus is selected from the group consisting of: DSM22592, DSM22585, DSM18111, DSM21408, DSM22587, DSM22884, CNCM I-3617 (WO2008/040734), DSM18344 (WO2007/144770), CNCM I-2980 (US2006/0240539) and mutants and variants of any of these strains.

In a final aspect, the present invention relates to a fermented milk product obtainable by the method of the invention.

In an interesting embodiment, the fermented milk product of the invention comprises an ingredient selected from the group consisting of: a fruit concentrate, a syrup, a probiotic bacterial culture (e.g. a culture of a Bifidibacterium; e.g. BB-12 ®), a prebiotic agent, a coloring agent, a thickening agent, a flavoring agent, and a preserving agent.

The fermented milk product of the invention may be in the form of a stirred type product, a set type product, or a drinkable product. The fermented milk product of the invention may also be in the form of a cheese, e.g. fromage frais.

### DEFINITIONS

In the present context, the term “milk substrate” may be any raw and/or processed milk material that can be subjected to fermentation according to the method of the invention. Thus, useful milk substrates include, but are not limited to, solutions/suspensions of any milk or milk like products comprising protein, such as whole or low fat milk, skim milk, buttermilk, reconstituted milk powder, condensed milk, dried milk, whey, whey permeate, lactose, mother liquid from crystallization of lactose, whey protein concentrate, or cream. Obviously, the milk substrate may originate from any mammal, e.g. being substantially pure mammalian milk, or reconstituted milk powder.

Preferably, at least part of the protein in the milk substrate is proteins naturally occurring in milk, such as casein or whey protein. However, part of the protein may be proteins which are not naturally occurring in milk.

The term “milk” is to be understood as the lacteal secretion obtained by milking any mammal, such as cows, sheep, goats, buffaloes or camels. In a preferred embodiment, the milk is cow's milk.

Prior to fermentation, the milk substrate may be homogenized and pasteurized according to methods known in the art.

“Homogenizing” as used herein means intensive mixing to obtain a soluble suspension or emulsion. If homogenization is performed prior to fermentation, it may be performed so as to break up the milk fat into smaller sizes so that it no longer separates from the milk. This may be accomplished by forcing the milk at high pressure through small orifices.

“Pasteurizing” as used herein means treatment of the milk substrate to reduce or eliminate the presence of live organisms, such as microorganisms. Preferably, pasteurization is attained by maintaining a specified temperature for a specified period of time. The specified temperature is usually attained by heating. The temperature and duration may be selected in order to kill or inactivate certain bacteria, such as harmful bacteria. A rapid cooling step may follow.

“Fermentation” in the methods of the present invention means the conversion of carbohydrates into alcohols or acids through the action of a microorganism (such as a lactic acid bacterium, e.g. of the species Lactobacillus sp. and Streptococcus thermophilus). Preferably, fermentation in the methods of the invention comprises conversion of lactose to lactic acid.

Lactic acid bacteria, including bacteria of the species Lactobacillus sp. and Streptococcus thermophilus, are normally supplied to the dairy industry either as frozen or freeze-dried cultures for bulk starter propagation or as so-called “Direct Vat Set” (DVS) cultures, intended for direct inoculation into a fermentation vessel or vat for the production of a dairy product, such as a fermented milk product. Such cultures are in general referred to as “starter cultures” or “starters”. In the present context, a “fermented milk product”, or “fermented milk”, should be understood as a milk substrate subjected to fermentation by bacteria of species Lactobacillus (especially Lactobacillus fermentum), optionally together with bacteria of the species Streptococcus thermophilus. Optionally, the fermented milk (product) may be subjected to heat treatment to inactivate the bacteria.

Fermentation processes to be used in production of fermented milk products are well known and the person of skill in the art will know how to select suitable process conditions, such as temperature, oxygen, addition of carbohydrates, amount and characteristics of microorganism(s) and process time. Obviously, fermentation conditions are selected so as to support the achievement of the present invention, i.e. to obtain a fermented milk product.

The term “stirred type product” specifically refers to a fermented milk product which sustains a mechanical treatment after fermentation, resulting in a destructuration and liquefaction of the coagulum formed under the fermentation stage. The mechanical treatment is typically but not exclusively obtained by stirring, pumping, filtrating or homogenizing the gel, or by mixing it with other ingredients. Stirred type products typically but not exclusively have a milk solid non-fat content of 9 to 15%.

The term “set-type product” includes a product based on milk which has been inoculated with a starter culture, e.g. a starter culture, and packaged next to the inoculating step and then fermented in the package.

The term “drinkable product” includes beverages such as “drinking yoghurt” and similar. The term “drinking yoghurt” typically covers a milk product produced by fermentation by the combination of Lactobacillus species and Streptococcus thermophilus. Drinking yoghurt typically has a milk solid non-fat content of 8% or more. Furthermore, the live culture count for drinking yoghurt drinks is typically at least 10E6 cell forming units (CFU) pr ml.

In the present context, the term “mutant” should be understood as a strain derived from a strain of the invention by means of e.g. genetic engineering, radiation and/or chemical treatment. It is preferred that the mutant is a functionally equivalent mutant, e.g. a mutant that has substantially the same, or improved, properties (e.g. regarding viscosity, gel stiffness, mouth coating, flavor, and/or post acidification) as the mother strain. Such a mutant is a part of the present invention. Especially, the term “mutant” refers to a strain obtained by subjecting a strain of the invention to any conventionally used mutagenization treatment including treatment with a chemical mutagen such as ethane methane sulphonate (EMS) or N-methyl-N′-nitro-N-nitroguanidine (NTG), UV light or to a spontaneously occurring mutant.

In the present context, the term “variant” should be understood as a strain which is functionally equivalent to a strain of the invention, e.g. having substantially the same, or improved, properties (e.g. regarding viscosity, gel stiffness, mouth coating, flavour, and/or post acidification). Such variants, which may be identified using appropriate screening techniques, are a part of the present invention.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising”, “having”, “including” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

### EXAMPLES

### Example 1

### Comparison of Fermented Milks Produced with Streptococcus thermophilus+the Lactobacillus Species fermentum with Traditional Yoghurts Produced with Streptococcus thermophilus+Lactobacillus delbruckii subsp. bulgaricus

Four different strains of Streptococcus thermophilus were applied (one by one) in order to get a general view of the Lactobacillus properties—irrespectively of the selection of Streptococcus thermophilus strain (hereafter named: ST-strain).

12 fermented milks were produced in 200 mL scale in duplicate. Lactobacillus strains fermentum (n=1) and delbrueckii subsp. bulgaricus (n=2) were tested one by one in combination with 4 different ST-strains strains each. Five percent of ST-strain CHCC7018 (DSM21408) was added to each culture to ensure a sufficient acidification rate.

The milk base consisted of milk with 1.5% fat, added 2% skimmed milk powder and 5% sucrose. The milk base was heat-treated 20 min. at 90 deg. C. and cooled to the fermentation temperature 40 deg. C. Hereafter it was inoculated with 0.02% lactic acid bacteria culture (F-DVS=Frozen Direct Vat Set culture). The culture compositions appear in table 1. After fermentation to pH 4.55 the yoghurts were stirred in a standardized way, cooled in water bath to 25 deg. C. and stored at 8 deg C. until analyses were performed at respectively day 1 and day 7.

TABLE 1
Culture compositions used for the example study.

| | Lactobacillus | | | | | | | |
|---|---|---|---|---|---|---|---|---|
| | | delbrueckii | delbrueckii | | | | | |
| | | subsp. | subsp. | | | | | |
| | fermentum | bulgaricus | bulgaricus | Streptococcus thermophilus | | | | |
| DSM | 22584 | 17959 | 22586 | * | 22592 | 22585 | 18111 | 21408 |
| CHCC | 2008 | 7159 | 4351 | | 10655 | 4239 | 6008 | 7018 |
| 101 | 30% | | | 65% | | | | 5% |
| 102 | 30% | | | | 65% | | | 5% |
| 103 | 30% | | | | | 65% | | 5% |
| 104 | 30% | | | | | | 65% | 5% |
| 113 | | 30% | | 65% | | | | 5% |
| 114 | | 30% | | | 65% | | | 5% |
| 115 | | 30% | | | | 65% | | 5% |
| 116 | | 30% | | | | | 65% | 5% |
| 117 | | | 30% | 65% | | | | 5% |
| 118 | | | 30% | | 65% | | | 5% |
| 119 | | | 30% | | | 65% | | 5% |
| 120 | | | 30% | | | | 65% | 5% |

* Commercial product ST-BODY-4 which is available from Chr. Hansen A/S

The pH was measured after respectively 1 and 7 days storage. As all the products were fermented to the same end pH (4,55), the pH after storage reflects the level of post acidification that has taken place during storage.

The rheological analyses were performed using a StressTech rheometer from Rheologica Instruments, Lund, Sweden. The analyses were performed at 13 deg. C. Initially, G*, reflecting Gel Stiffness, was measured by oscillation at frequency 1 Hz. Subsequently a flow curve measuring the shear stress as a function of shear rates from 0 1/s to 300 1/s to 0 1/s (in an up and down sweep) was recorded. Hysteresis loop area between the up- and down curves were calculated and divided with area under upper curve—to provide the relative loop area. The shear stress measured at shear rate 300 1/s was chosen to represent the apparent viscosity of the samples (data recorded in table 2). See FIG. 1 for example of flow curves.

Volatile compounds (VOC) derived from the fermentation of bacterial cultures in milk were analysed by Static Head Space Gas Chromatography (HSGC) using a Autosystem XL GC fitted with a flame ionization detector (Perkin Elmer, Waltham, US). The fermented milk samples were conserved by solid NaCl, NaF and stabilized with phosphate buffer (pH 7). A part of the Head Space (50 μL) was injected into the GC, which separates the volatile compounds according to chemical nature and boiling point. Peak size (height and area) is directly proportional to the compound concentration in the injected sample volume. By analyzing standards (known concentration) response factors (response per peak size) were determined and peak sizes obtained from the analysed samples were converted to ppm using response factors.

TABLE 2
Results from example study. All data are averages over 4 products per
Lactobacillus strain (4 different ST-strains) and 2 replicates.

| | | | | Gel | |
|---|---|---|---|---|---|
| | pH | pH | Shear stress | stiffnes | Loop area |
| | (day 1) | (day 7) | (Pa) | G* (Pa) | (relative) |
| Lb. fermentum | 4.51 | 4.38 | 92.3 | 94.3 | 0.38 |
| DSM22584 | | | | | |
| CHCC2008 | | | | | |
| Lb. bulgaricus | 4.39 | 4.30 | 88.8 | 87.5 | 0.38 |
| DSM17959 | | | | | |
| CHCC7159 | | | | | |
| Lb. bulgaricus | 4.42 | 4.35 | 89.8 | 79.8 | 0.39 |
| DSM22586 | | | | | |
| CHCC4351 | | | | | |

Fermented milks with the Lactobacillus species fermentum have higher pH values after 1 and 7 days of storage compared to the products with Lb. delbrueckii subsp. bulgaricus. This means that a lower level of post acidification takes place in these fermented milks compared to the classical yoghurts with Lb. delbruckii subsp. bulgaricus (also called Lb. bulgaricus). Low post acidification is a very valuable property as it enables production of mild fermented milk products which are requested by most consumers.

Higher viscosities (shear stress) were obtained in the fermented milks produced with Lb. fermentum compared to Lb. bulgaricus. FIG. 1 shows flow curves for fermented milks produced with Lb. fermentum and 2 different bulgaricus strains—all in the same background (combination with CHCC6008 and CHCC7018). The apparent viscosities (shear stress levels) are clearly higher for the products with fermentum compared to the two products with bulgaricus. This applies for all shear rates from 50 1/s and up to 300 1/s.

Very interestingly, the products with Lb. fermentum also obtained higher gel stiffness levels than did the 2 products with Lb. bulgaricus. It is unusual to see this combined effect (higher viscosity and higher gel stiffness) resulting from a lactic acid bacteria culture. Often, improved viscosity results in reduced gel stiffness. However, the combination of high viscosity and high gel stiffness is commercially very attractive as described in the background section.

The last rheological parameter ‘loop area’ does not seem to be affected by the choice of Lactobacillus species.

In conclusion, the study shows that applying the Lactobacillus species fermentum enables production of fermented milk products which are milder (lower post acidification) and have higher viscosity as well as higher gel stiffness compared to products produced with Lb. bulgaricus—in the same culture background (four different background cultures tested).

The fermented milk product prepared using an Streptococcus thermophilus stain and a Lactobacillus strain of the invention was analysed regarding volatile compounds, and compared to standard yoghurts (see table 3). Large differences in volatile compounds were found between the fermented milk product produced with Lb. fermentum compared to the other fermented milk products. Interestingly, a remarkably higher ethanol level was found in the fermented milk products containing Lb. fermentum. In addition, acetaldehyde and acetone levels were lower for the fermented milk prepared with Lb. fermentum. It is suggested that these differences in volatile compounds contributes to the flavor and mouthfeel of the ‘yoghurts’.

TABLE 3
Results from example study. All data are averages over 4 products per
Lactobacillus strain (4 different ST-strains) and 2 replicates.

| | Acetaldehyde | Acetone | Ethanol | Diacetyl |
|---|---|---|---|---|
| Sample | (ppm) | (ppm) | (ppm) | (ppm) |
| Lb. fermentum | 2.9 | 0.6 | 10.6 | <0.1 |
| CHCC2008 | | | | |
| Lb. bulgaricus | 6.6 | 1.2 | <2.9 | <0.1 |
| CHCC7159 | | | | |
| Lb. bulgaricus | 7.1 | 1.2 | <2.9 | <0.1 |
| CHCC4351 | | | | |

### Example 2

### Effect of Lactobacillus fermentum DSM 22584 in Low Fat Yoghurt

2 fermented milks were produced in 3 L scale. Lactobacillus fermentum DSM 22584 was tested in combination with a blend of 2 different Streptococcus thermophilus strains (DSM22587 and DSM 22884) and presence of Lactobacillus delbruckii subsp. bulgaricus strain DSM 19252. The control culture contained only the same two ST strains and Lactobacillus delbruckii subsp. bulgaricus dsm19252 (see table 4).

The milk base consisted of skimmed milk added 2% skimmed milk powder. The milk base was heat-treated 6 min. at 95 deg. C. and cooled to the fermentation temperature 42 deg. C. Hereafter it was inoculated with 0.018% lactic acid bacteria culture (F-DVS=Frozen Direct Vat Set culture). The culture compositions appear in table 3. After fermentation to pH 4.55 a mechanical post treatment was applied (42 deg.0/2 bar/flow 45 l/hour) during 1 minutes and the yoghurts were cooled to 5 deg. C. and stored at 5 deg C. until analyses were performed at respectively day 4 and day 35.

TABLE 4
Culture composition used for the example study.

| | | Lactobacillus | | |
|---|---|---|---|---|
| | Lactobacillus | delbrueckii susp. | Streptococcus | |
| | fermentum | bulgaricus | thermophilus | |
| DSM | 22584 | 19252 | 22587 | 22884 |
| CHCC | 2008 | 10019 | 5086 | 11379 |
| M1 | 11% | 5.5% | 72.5% | 11% |
| Control | — | 11% | 78% | 11% |

The pH was measured at 35 days storage. As all the products were fermented to the same end pH (4,55), the pH after storage reflects the level of post acidification that has taken place during storage.

Qualitative sensory evaluation was made by 5 experts at day 4 after production.

The rheological analyses were performed as in example 1.

TABLE 5
Results from example study 2.

| | | | | | Sensory evaluation | |
|---|---|---|---|---|---|---|
| | | Rheology | | | | Mouth |
| | pH | Shear | | | Thickness | coating |
| | pH | stress | | | Force to | Yoghurt |
| | decrease | (Pa) at | | | take the | stay in |
| | from | shear | Gel | Loop | product up | mouth |
| | day 0 to | rate 300 | stiffnes | area | with a | after |
| | day 35 | 1/s | G* (Pa) | (relative) | spoon | swallowing |
| Lb. fermentum | 0.16 | 48.0 | 32.66 | 0.38 | + | + |
| M1 | | | | | | |
| DSM22584 | | | | | | |
| CHCC2008 | | | | | | |
| Control | 0.18 | 45.8 | 4.10 | 0.37 | − | − |

The use of Lactobacillus species fermentum in the yoghurt did not increase post acidification.

The apparent viscosity (shear stress levels) is slightly higher for the products with fermentum compared to the control. The ‘loop area’ was not affected by the culture choice.

Surprisingly, the products with Lb. fermentum obtained significantly higher gel stiffness levels than did the control product with Lb. bulgaricus as single Lactobacillus species. It is unusual to see this combined effect (higher viscosity and higher gel stiffness) resulting from a lactic acid bacteria culture. Often, improved viscosity results in reduced gel stiffness. However, the combination of high viscosity and high gel stiffness is commercially very attractive as described in the background section.

The impact of Lb. fermentum on gel stiffness was clearly found by the assessors in sensory analysis.

In conclusion, the study shows that applying the Lactobacillus species fermentum enables production of fermented milk products which are mild (low post acidification), showing high viscosity and at the same time significantly higher gel stiffness compared to products produced with Lb. bulgaricus as single Lactobacillus species in combination with the same Streptococcus thermophilus strains.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

### DRAWING

FIG. 1 depicts the flow curves for fermented milks, measuring shear stress as a function of shear rate, for fermented milk samples 104 (Lb. fermentum CHCC2008+ST (CHCC6008+CHCC7018), 116 (Lb. bulgaricus CHCC7159+ST (CHCC6008+CHCC7018) and 120 (Lb. bulgaricus CHCC4351+ST (CHCC6008+CHCC7018)

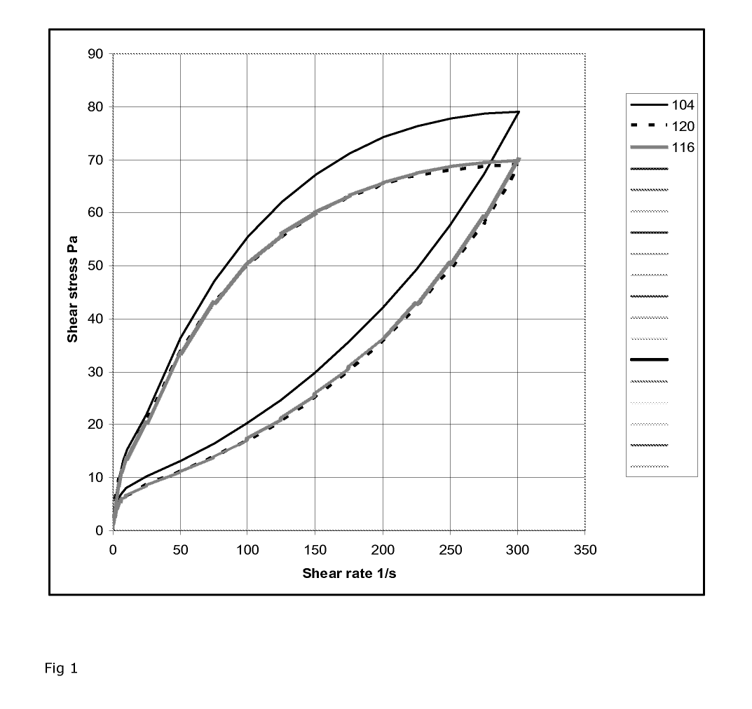

### DEPOSITS AND EXPERT SOLUTION

The applicant requests that a sample of the deposited microorganisms stated below may only be made available to an expert approved by the applicant. The deposits were made according to the Budapest treaty on the international recognition of the deposit of microorganisms for the purposes of patent procedure.

The Lactobacillus and Streptococcus strains were deposited 19 May 2009 at Deutsche Sammlung von Mikroorganismen and Zellkulturen GmbH, Inhoffenstr. 7B, D-38124 Braunschweig (DSMZ) and given the accession numbers:

Lb. fermentum CHCC2008: DSM22584
Lb. delbrueckii subsp bulgaricus CHCC4351: DSM22586
Streptococcus thermophilus CHCC10655: DSM22592
Streptococcus thermophilus CHCC4239: DSM22585
Further deposits at DSMZ:
Lb. bulgaricus CHCC7159: DSM17959 (date of deposit: 8 Feb. 2006)
Streptococcus thermophilus CHCC6008: DSM18111 (date of deposit: 23 Mar. 2006)
Streptococcus thermophilus CHCC7018: DSM21408 (date of deposit: 23 Apr. 2008)
Streptococcus thermophilus CHCC5086: DSM22587 (date of deposit: 19 May 2009)
Streptococcus thermophilus CHCC11379: DSM22884 (date of deposit 26 Aug. 2009)
Lb. delbrueckii subsp: bulgaricus CHCC10019: DSM19252 (date of deposit 3 Apr. 2007)

### REFERENCES

WO2008/040734, WO2007/144770, US2006/0240539, WO2007/147890, WO2010/023290.

All references cited in this patent document are hereby incorporated herein in their entirety by reference.

1. A method for producing a fermented milk product, comprising fermenting a milk substrate with a strain belonging to a Lactobacillus species, which is able to produce a polysaccharide (such as a homopolysaccharide or a heteropolysaccharide) and/or a glycosyltransferase (e.g. fructosyl transferase or glucosyl transferase) enzyme.

2. A method for producing a fermented milk product, comprising fermenting a milk substrate with a strain belonging to the species Lactobacillus fermentum.

3. The method of claim 1, further comprising fermenting the milk substrate with a strain belonging to the species: Streptococcus thermophilus, such as a polysaccharide producing strain, and/or a strain selected from the group consisting of: DSM22592, DSM22585, DSM18111, DSM21408, CNCM I-3617, DSM18344, DSM22587, DSM22884 and CNCM I-2980, and mutants and variants of any of these strains.

4. The method of claim 1, wherein the milk substrate is fermented with a strain belonging to the species Streptococcus thermophilus before, during, or after the fermentation with a strain belonging to a polysaccharide producing Lactobacillus species, a strain belonging to a glycosyltransferase enzyme producing Lactobacillus species, and/or a strain belonging to the species Lactobacillus fermentum.

5. The method of claim 1, wherein the milk substrate is fermented with a strain belonging to the species Streptococcus thermophilus during the fermentation with a strain belonging to a polysaccharide producing Lactobacillus species, a strain belonging to a glycosyltransferase enzyme producing Lactobacillus species, and/or a strain belonging to the species Lactobacillus fermentum.

6. The method of claim 1, comprising adding an enzyme to the milk substrate before, during and/or after the fermenting, such as an enzyme selected from the group consisting of: an enzyme able to crosslink proteins, transglutaminase, an aspartic protease, chymosin, and rennet.

7. The method of claim 1, wherein the Lactobacillus species is Lactobacillus fermentum.

8. The method of claim 1, wherein the strain belonging to a Lactobacillus species is selected from the group consisting of Lactobacillus fermentum DSM22584, and mutants and variants of this strain.

9. The method of claim 1, wherein the milk substrate is fermented with a strain belonging to the species Lactobacillus delbrueckii subsp. bulgaricus or lactis, in addition to the strain belonging to a Lactobacillus species, which is able to produce a polysaccharide and/or a glycosyltransferase enzyme; and/or to the strain belonging to the species Lactobacillus fermentum.

10. The method according to claim 9, wherein
a) the strain belonging to the species Lactobacillus bulgaricus or Lactobacillus lactis, and
b) the strain belonging to a Lactobacillus species, which is able to produce a polysaccharide and/or a glycosyltransferase enzyme and/or the strain belonging to the species Lactobacillus fermentum,
are added in a ratio (measured in CFU/g milk substrate) within the range 1/100 to 100/1 (a/b).

11. The method of claim 1, wherein the ratio (measure in CFU/g milk substrate) between bacteria belonging to a Lactobacillus species, and bacteria belonging to a Streptococcus species, is within the range 1/100 to 100/1.

12. The method of claim 1, wherein glucose or sucrose is added to the milk substrate (and/or the milk substrate comprises glucose or sucrose), such as in an amount of at least 1 gram per liter.

13. A fermented milk product obtainable by a method of claim 1.

14. The fermented milk product of claim 13, which comprises an ingredient selected from the group consisting of: a fruit concentrate, a syrup, a probiotic bacterial culture, prebiotic agent, a coloring agent, a thickening agent, a flavoring agent, and a preserving agent.

15. The fermented milk product of claim 13, which is in the form of a stirred type product, a set type product, or a drinkable product.

16. A strain belonging to a polysaccharide (e.g. homopolysaccharide or heteropolysaccharide) producing Lactobacillus species, said strain comprises the nucleotide sequence encoding a glycosyltransferase (e.g. fructosyl transferase or glucosyl transferase) enzyme, and/or the strain produces a glycosyltransferase (e.g. fructosyl transferase or glucosyl transferase) enzyme.

17. A bacterial strain selected from the group consisting of Lactobacillus fermentum DSM22584, and mutants and variants of this strain.

18. A bacterial strain belonging to the species Streptococcus thermophilus, selected from the group consisting of: DSM22592, DSM22585, DSM18111, DSM21408, DSM22587 and DSM22884, and mutants and variants of any of these.

19. A composition comprising, either as a mixture or as a kit-of-parts,
a strain belonging to a polysaccharide (such as a homopolysaccharide or a heteropolysaccharide) and/or glycosyltransferase (e.g. fructosyl transferase or glucosyl transferase) enzyme producing Lactobacillus species and/or a Lactobacillus fermentum strain (such as DSM22584, or a mutant or a variant of this strain); and
a strain belonging to the species Streptococcus thermophilus (such as a polysaccharide producing strain).

20. The composition of claim 19, which comprises at least 10exp7 CFU/g (cell forming units per gram), such as at least 10exp8 or 10exp10 CFU/g, of a strain belonging to the polysaccharide and/or glycosyltransferase enzyme producing Lactobacillus species and/or the Lactobacillus fermentum strain; and at least 10exp7 CFU/g, such as at least 10exp8 or 10exp10 CFU/g, of the strain belonging to the species Streptococcus thermophilus.

21. The composition of claim 1, which is usable as a starter culture, and is in frozen, freeze-dried or liquid form.

22. The composition of claim 1, wherein the strain belonging to the Lactobacillus species is selected from the group consisting of Lactobacillus fermentum DSM22584 and mutants or variants of this strain; and the strain belonging to the species Streptococcus thermophilus is selected from the group consisting of: DSM22592, DSM22585, DSM18111, DSM21408, DSM22587, DSM22884, CNCM I-3617, DSM18344, CNCM I-2980, and mutants and variants of any of these strains.

23. A composition comprising, either as a mixture or as a kit-of-parts, a bacterial strain defined in claim 17 and a bacterial strain belonging to the species Streptococcus thermophilus, selected from the group consisting of: DSM22592, DSM22585, DSM18111, DSM21408, DSM22587 and DSM22884, and mutants and variants of any of these.

24. A fermented milk product obtainable by adding a composition of claim 19, or a bacterial strain of claim 17 to a milk substrate.

25. The fermented milk product of claim 24, which optionally comprises an ingredient selected from the group consisting of: a fruit concentrate, a syrup, a probiotic bacterial culture, prebiotic agent, a coloring agent, a thickening agent, a flavoring agent, and a preserving agent; and/or which optionally is in the form of a stirred type product, a set type product, or a drinkable product.
